(19) United States
(12) Patent Application Publication
(10) Pub. No.: US 2025/0095827 A1
(43) Pub. Date: Mar. 20, 2025

(54) METHOD FOR DETECTING CLARITY OF THYROID NODULE BOUNDARY, SYSTEM THEREOF, ELECTRONIC DEVICE AND MEDIUM

(71) Applicant: TenD.AI Medical Technology (Shanghai) Co., Ltd., Shanghai (CN)

(72) Inventor: Jun GUO, Shanghai (CN)

(21) Appl. No.: 18/679,951

(22) Filed: May 31, 2024

(30) Foreign Application Priority Data
Sep. 15, 2023 (CN) 202311196676.8

(51) Int. Cl. G16H 30/40

(52) U.S. Cl. CPC G16H 30/40

(57) ABSTRACT

A method for detecting clarity of a thyroid nodule boundary, a system thereof, an electronic device and a medium are provided. The method includes: calculating an aspect ratio coefficient; calculating an inner and outer ring difference coefficient according to an intensity average of an outer ring image and an inner ring image; segmenting the outer ring image and the inner ring image into four parts; obtaining a four-partitioning intensity difference coefficient according to the intensity average of each segmented outer ring image and each segmented inner ring image; inputting a preprocessed image into a trained Thy-Enet deep neural network to obtain the probability that the thyroid nodule boundary is clear; and inputting the aspect ratio coefficient, the inner and outer ring difference coefficient, the four-partitioning intensity difference coefficient and the probability into a trained multi-layer perceptron model to obtain a determination result of the thyroid nodule boundary.

### CROSS-REFERENCE TO RELATED APPLICATION

This patent application claims the benefit and priority of Chinese Patent Application No. 2023111966768 filed with the China National Intellectual Property Administration on Sep. 15, 2023, the disclosure of which is incorporated by reference herein in its entirety as part of the present application.

### TECHNICAL FIELD

The present disclosure relates to the technical field of image processing, and in particular to a method of detecting clarity of a thyroid nodule boundary, a system thereof, an electronic device and a medium.

### BACKGROUND

A thyroid nodule is one of the most common thyroid problems. Ultrasonic imaging examination can quickly and accurately find and locate a thyroid nodule. Thyroid nodules can be detected in more than 20% of people through ultrasonic imaging examination. According to an ultrasonic image of a nodule cross section, doctors can judge the nature and development expectation of a nodule, and then make a follow-up diagnosis and treatment plan. In the ultrasonic features of the nodule, the clarity of the nodule boundary is one of the important indexes. A nodule with a clear boundary is often associated with a benign nodule, while a nodule with unclear boundary is often invasive, may develop into a malignant tumor, or has the characteristics of a malignant tumor.

An ultrasonic image has a limited resolution and an inherent speckle noise, so that it is difficult to judge the clarity of the nodule boundary in the ultrasonic image, which is highly dependent on the experience of doctors, and thus it is impossible to form specific standards. However, a method based on computer image processing and computer vision can characterize the image clarity, and help doctors to make judgments by setting thresholds. However, the existing methods are often only based on a single imaging measurement index, and do not refer to the actual judgment experience of doctors, and thus have limited ability to measure the nodule clarity, so that the final clarity result is inaccurate.

### SUMMARY

The present disclosure aims to provide a method for detecting clarity of a thyroid nodule boundary, a system thereof, an electronic device and a medium, which can improve the accuracy of the clarity result of the thyroid nodule boundary.

In order to achieve the above objectives, the present disclosure provides the following scheme.

A method for detecting clarity of a thyroid nodule boundary includes:
acquiring a target thyroid nodule ultrasonic image, where a thyroid nodule boundary is labelled in the target thyroid nodule ultrasonic image in a form of dots;
obtaining a bounding rectangle for the thyroid nodule boundary labelled in the target thyroid nodule ultrasonic image, and calculating an aspect ratio coefficient of the target thyroid nodule ultrasonic image according to a width of the bounding rectangle and a height of the bounding rectangle;
outwardly expanding and inwardly retracting a bounding polygon of the thyroid nodule boundary labelled in the target thyroid nodule ultrasonic image, to obtain an outwardly expanded boundary and an inwardly retracted boundary, where the bounding polygon of the thyroid nodule boundary labelled in the target thyroid nodule ultrasonic image is obtained by connecting all points in the thyroid nodule boundary labelled in the target thyroid nodule ultrasonic image counterclockwise or clockwise in sequence;
calculating an inner and outer ring difference coefficient of the target thyroid nodule ultrasonic image according to an intensity average of an outer ring image and an intensity average of an inner ring image; where the outer ring image is a first partial target thyroid nodule ultrasonic image between the outwardly expanded boundary and the bounding polygon of the thyroid nodule boundary labelled in the target thyroid nodule ultrasonic image; the inner ring image is a second partial target thyroid nodule ultrasonic image between the inwardly retracted boundary and the bounding polygon of the thyroid nodule boundary labelled in the target thyroid nodule ultrasonic image;
segmenting the outer ring image and the inner ring image into four parts equally in a same segmenting manner to obtain four segmented outer ring images and four segmented inner ring images;
obtaining a four-partitioning intensity difference coefficient of the target thyroid nodule ultrasonic image according to the intensity average of each segmented outer ring image and the intensity average of each segmented inner ring image;
inputting a preprocessed image into a trained Thy-Enet deep neural network to obtain a probability that the thyroid nodule boundary of the target thyroid nodule ultrasonic image is clear, where the preprocessed image is a target thyroid nodule ultrasonic image in an outwardly expanded target bounding rectangle, the outwardly expanded target bounding rectangle is obtained by outwardly expanding the bounding rectangle of the thyroid nodule boundary labelled in the target thyroid nodule ultrasonic image, the trained Thy-Enet deep neural network includes an input layer, an EfficientNetV2B0 backbone, a global pooling layer, a dropout layer and a Sigmoid function which are connected in sequence;
inputting the aspect ratio coefficient of the target thyroid nodule ultrasonic image, the inner and outer ring difference coefficient of the target thyroid nodule ultrasonic image, the four-partitioning intensity difference coefficient of the target thyroid nodule ultrasonic image and the probability that the thyroid nodule boundary of the target thyroid nodule ultrasonic image is clear, into a trained multi-layer perceptron model, to obtain a final probability that the thyroid nodule boundary of the target thyroid nodule ultrasonic image is clear; and
obtaining a determination result of the thyroid nodule boundary of the target thyroid nodule ultrasonic image according to the final probability that the thyroid nodule boundary of the target thyroid nodule ultrasonic image is clear, where the determination result is that thyroid nodule boundary is clear or unclear.

In this embodiment, the calculating an aspect ratio coefficient of the target thyroid nodule ultrasonic image according to a width of the bounding rectangle and a height of the bounding rectangle includes:
calculating a ratio of the height of the bounding rectangle to the width of the bounding rectangle to obtain an aspect ratio;
obtaining the aspect ratio coefficient of the target thyroid nodule ultrasonic image according to the aspect ratio.

In this embodiment, the calculating an inner and outer ring difference coefficient of the target thyroid nodule ultrasonic image according to an intensity average of an outer ring image and an intensity average of an inner ring image includes:

calculating an absolute value of a difference between the intensity average of the outer ring image and the intensity average of the inner ring image to obtain the inner and outer ring difference coefficient of the target thyroid nodule ultrasonic image.

In this embodiment, the segmenting the outer ring image and the inner ring image into four parts equally in a same segmenting manner to obtain four segmented outer ring images and four segmented inner ring images includes:
setting a center of the outer ring image as an origin, segmenting the outer ring image into four parts equally in counterclockwise direction according to angle ranges from 0 to 90 degrees, from 90 to 180 degrees, from 180 to 270 degrees and from 270 to 360 degrees, to obtain four segmented outer ring images;
setting a center of the inner ring image as an origin, segmenting the inner ring image into four parts equally in counterclockwise direction according to angle ranges from 0 to 90 degrees, from 90 to 180 degrees, from 180 to 270 degrees and from 270 to 360 degrees, to obtain four segmented inner ring images.

In this embodiment, the obtaining a four-partitioning intensity difference coefficient of the target thyroid nodule ultrasonic image according to the intensity average of each segmented outer ring image and the intensity average of each segmented inner ring image includes:
calculating the difference between an intensity average of a first segmented outer ring image and an intensity average of a first segmented inner ring image to obtain a first difference;
where a position of the first segmented outer ring image in the outer ring image is same as that of the first segmented inner ring image in the inner ring image;
calculating a difference between an intensity average of a second segmented outer ring image and an intensity average of a second segmented inner ring image to obtain a second difference; where a position of the second segmented outer ring image in the outer ring image is same as that of the second segmented inner ring image in the inner ring image;
calculating a difference between an intensity average of a third segmented outer ring image and an intensity average of a third segmented inner ring image to obtain a third difference; where a position of the third segmented outer ring image in the outer ring image is same as that of the third segmented inner ring image in the inner ring image;
calculating a difference between an intensity average of a fourth segmented outer ring image and an intensity average of a fourth segmented inner ring image to obtain a fourth difference;
where a position of the fourth segmented outer ring image in the outer ring image is a same as that of the fourth segmented inner ring image in the inner ring image;
determining a minimum value among the first difference, the second difference, the third difference and the fourth difference as the four-partitioning intensity difference coefficient of the target thyroid nodule ultrasonic image.

In this embodiment, the trained Thy-Enet depth neural network is determined by a following process:
acquiring a sample set, where the sample set includes a plurality of sample thyroid nodule ultrasonic images and a true probability that a thyroid nodule boundary of each of the sample thyroid nodule ultrasonic images is clear; the thyroid nodule boundary is labelled in each sample thyroid nodule ultrasonic image;
performing data enhancement processing on each sample thyroid nodule ultrasonic image in the sample set to obtain a plurality of enhanced sample thyroid nodule ultrasonic images, where one sample thyroid nodule ultrasonic image corresponds to one enhanced sample thyroid nodule ultrasonic image;
training a Thy-Enet deep neural network according to the plurality of enhanced sample thyroid nodule ultrasonic images and the true probability that the thyroid nodule boundary of each of the sample thyroid nodule ultrasonic images is clear to obtain the trained Thy-Enet deep neural network.

In this embodiment, the trained multi-layer perceptron model is determined by a following process:
acquiring a determination result of the thyroid nodule boundary of each of the sample thyroid nodule ultrasonic images;
determining an aspect ratio coefficient of each of the sample thyroid nodule ultrasonic images, an inner and outer ring difference coefficient of each of the sample thyroid nodule ultrasonic images, the four-partitioning intensity difference coefficient of each of the sample thyroid nodule ultrasonic images and the probability that the thyroid nodule boundary of each of the sample thyroid nodule ultrasonic images is clear; the probability that the thyroid nodule boundary of the sample thyroid nodule ultrasonic image is clear is obtained by inputting a preprocessed sample image into the trained Thy-Enet deep neural network; the preprocessed sample image is a partial sample thyroid nodule ultrasonic image in an outwardly expanded sample bounding rectangle; and the outwardly expanded sample bounding rectangle is obtained by outwardly expanding a bounding rectangle of the thyroid nodule boundary of each sample thyroid nodule ultrasonic image;
training the multi-layer perceptron model according to the aspect ratio coefficient of each of the sample thyroid nodule ultrasonic images, the inner and outer ring difference coefficient of each of the sample thyroid nodule ultrasonic images, the four-partitioning intensity difference coefficient of each of the sample thyroid nodule ultrasonic images, the probability that the thyroid nodule boundary of each of the sample thyroid nodule ultrasonic images is clear, and the determination result of the thyroid nodule boundary of each of the sample thyroid nodule ultrasonic images to obtain the trained multi-layer perceptron model.

A system for detecting clarity of a thyroid nodule boundary includes:
an acquiring module, configured to acquire a target thyroid nodule ultrasonic image; where a thyroid nodule boundary is labelled in the target thyroid nodule ultrasonic image in a form of dots;
an aspect ratio coefficient calculating module, configured to obtain a bounding rectangle for the thyroid nodule boundary labelled in the target thyroid nodule ultrasonic image, and calculate an aspect ratio coefficient of the target thyroid nodule ultrasonic image according to a width of the bounding rectangle and a height of the bounding rectangle;
a boundary outwardly expanding and inwardly retracting module, configured to outwardly expand and inwardly retract a bounding polygon of the thyroid nodule boundary labelled in the target thyroid nodule ultrasonic image, to obtain an outwardly expanded boundary and an inwardly retracted boundary; where the bounding polygon of the thyroid nodule boundary labelled in the target thyroid nodule ultrasonic image is obtained by connecting all points in the thyroid nodule boundary labelled in the target thyroid nodule ultrasonic image counterclockwise or clockwise in sequence;
an inner and outer ring difference coefficient calculating module, configured to calculate an inner and outer ring difference coefficient of the target thyroid nodule ultrasonic image according to an intensity average of an outer ring image and an intensity average of an inner ring image; where the outer ring image is a first partial target thyroid nodule ultrasonic image between the outwardly expanded boundary and the bounding polygon of the thyroid nodule boundary labelled in the target thyroid nodule ultrasonic image; the inner ring image is a second partial target thyroid nodule ultrasonic image between the inwardly retracted boundary and the bounding polygon of the thyroid nodule boundary labelled in the target thyroid nodule ultrasonic image;
a segmenting module, configured to segment the outer ring image and the inner ring image into four parts equally in a same segmenting manner to obtain four segmented outer ring images and four segmented inner ring images;
a four-partitioning intensity difference coefficient calculating module, configured to obtain a four-partitioning intensity difference coefficient of the target thyroid nodule ultrasonic image according to the intensity average of each segmented outer ring image and the intensity average of each segmented inner ring image;
a clarity probability calculating module, configured to input a preprocessed image into a trained Thy-Enet deep neural network to obtain a probability that the thyroid nodule boundary of the target thyroid nodule ultrasonic image is clear; where the preprocessed image is a target thyroid nodule ultrasonic image in an outwardly expanded target bounding rectangle; the outwardly expanded target bounding rectangle is obtained by outwardly expanding the bounding rectangle of the thyroid nodule boundary labelled in the target thyroid nodule ultrasonic image; the trained Thy-Enet deep neural network includes an input layer, an EfficientNetV2B0 backbone, a global pooling layer, a dropout layer and a Sigmoid function which are connected in sequence;
a final clarity probability calculating module, configured to input the aspect ratio coefficient of the target thyroid nodule ultrasonic image, the inner and outer ring difference coefficient of the target thyroid nodule ultrasonic image, the four-partitioning intensity difference coefficient of the target thyroid nodule ultrasonic image and the probability that the thyroid nodule boundary of the target thyroid nodule ultrasonic image is clear, into a trained multi-layer perceptron model, to obtain a final probability that the thyroid nodule boundary of the target thyroid nodule ultrasonic image is clear;
a determination result calculating module, configured to obtain a determination result of the thyroid nodule boundary of the target thyroid nodule ultrasonic image according to the final probability that the thyroid nodule boundary of the target thyroid nodule ultrasonic image is clear; where the determination result is that thyroid nodule boundary is clear or unclear.

An electronic device according to an embodiment of the present disclosure includes:
a memory and a processor, where the memory is configured to store a computer program, and the processor is configured to run the computer program to cause the electronic device to execute the method for detecting clarity of the thyroid nodule boundary described above.

A computer-readable storage medium is provided, where a computer program is stored therein, and the computer program, when executed by a processor, implements the method for detecting clarity of the thyroid nodule boundary described above.

According to the specific embodiment of the present disclosure, the present disclosure provides the following technical effects.

According to the present disclosure, the trained Thy-Enet deep neural network is used to extract the probability that the thyroid nodule boundary is clear. Moreover, the aspect ratio coefficient, the inner and outer ring difference coefficient, the four-partitioning intensity difference coefficient and the probability that the thyroid nodule boundary is clear are input into a multi-layer perceptron model to obtain the final probability that the thyroid nodule boundary of the target thyroid nodule ultrasonic image is clear. The determination result is obtained according to the final probability, so that the accuracy of the clarity result of the thyroid nodule boundary can be improved, thereby assisting a sonographer to improve the ability of evaluating clarity of the thyroid nodule boundary.

### BRIEF DESCRIPTION OF THE DRAWINGS

In order to explain the embodiments of the present disclosure or the technical schemes in the prior art more clearly, the drawings that need to be used in the embodiments will be briefly introduced. Obviously, the drawings in the following description are only some embodiments of the present disclosure. For those skilled in the art, other drawings can be acquired according to these drawings without creative labor.

FIG. 1 is a flowchart of a method for detecting clarity of a thyroid nodule boundary according to an embodiment of the present disclosure.

FIG. 2 is a picture of a nodule with a boundary outlined.

FIG. 3 is a picture of a bounding rectangle of a nodule region.

FIG. 4 is a picture of a nodule boundary on which an outward expansion boundary and an inward retraction boundary of a bounding polygon are outlined.

FIG. 5 is a schematic diagram of a structure of a Thy-Enet deep neural network.

FIG. 6 is an outer ring image.

FIG. 7 is an inner ring image.

FIG. 8 is the outer ring image of FIG. 6 which is equally segmented.

FIG. 9 is a schematic diagram of a multi-layer perceptron model.

FIG. 10 is a flowchart of a more specific method for detecting clarity of a thyroid nodule boundary according to an embodiment of the present disclosure.

### DETAILED DESCRIPTION OF THE EMBODIMENTS

The technical schemes in the embodiments of the present disclosure will be clearly and completely described with reference to the drawings in the embodiments of the present disclosure hereinafter. Obviously, the described embodiments are only some embodiments of the present disclosure, rather than all of the embodiments. Based on the embodiment of the present disclosure, all other embodiments acquired by those skilled in the art without creative labor fall within the scope of protection of the present disclosure.

In order to make the above objects, features and advantages of the present disclosure more obvious and understandable, the present disclosure will be explained in further detail with reference to the drawings and detailed description hereinafter.

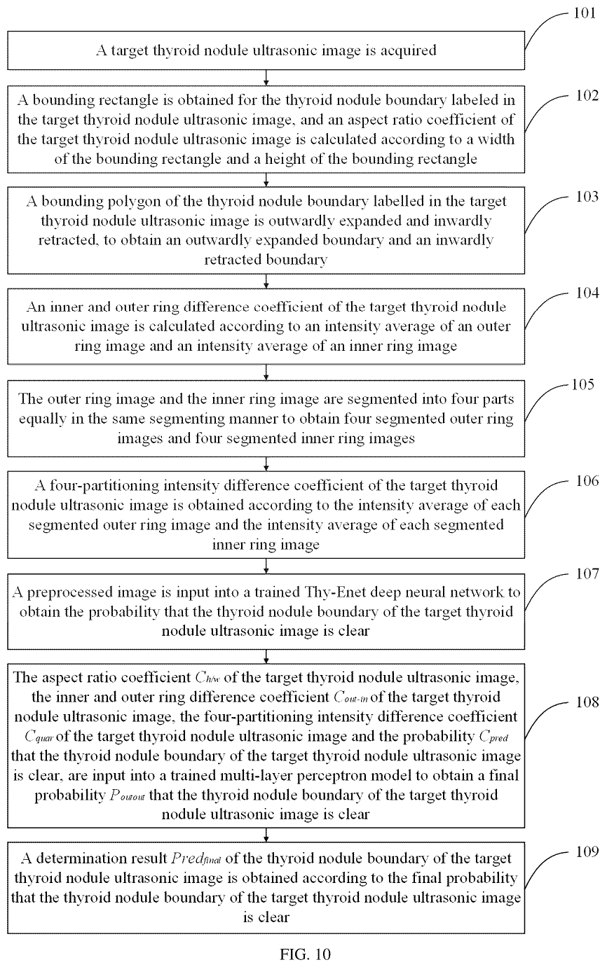

As shown in FIG. 10, a method for detecting clarity of a thyroid nodule boundary according to the embodiment of the present disclosure includes the following steps.

Step 101: a target thyroid nodule ultrasonic image is acquired. A thyroid nodule boundary is labelled in the target thyroid nodule ultrasonic image and is manually outlined by a doctor in a form of dots.

Step 102: a bounding rectangle is obtained for the thyroid nodule boundary labelled in the target thyroid nodule ultrasonic image, and an aspect ratio coefficient of the target thyroid nodule ultrasonic image is calculated according to a width of the bounding rectangle and a height of the bounding rectangle.

Step 103: a bounding polygon of the thyroid nodule boundary labelled in the target thyroid nodule ultrasonic image is outwardly expanded and inwardly retracted, to obtain an outwardly expanded boundary and an inwardly retracted boundary; where the bounding polygon of the thyroid nodule boundary labelled in the target thyroid nodule ultrasonic image is obtained by connecting all points in the thyroid nodule boundary labelled in the target thyroid nodule ultrasonic image counterclockwise or clockwise in sequence.

Step 104: an inner and outer ring difference coefficient of the target thyroid nodule ultrasonic image is calculated according to an intensity average of an outer ring image and an intensity average of an inner ring image. The outer ring image is a target thyroid nodule ultrasonic image between the outwardly expanded boundary and the bounding polygon of the thyroid nodule boundary labelled in the target thyroid nodule ultrasonic image; the inner ring image is a target thyroid nodule ultrasonic image between the inwardly retracted boundary and the bounding polygon of the thyroid nodule boundary labelled in the target thyroid nodule ultrasonic image.

Step 105: the outer ring image and the inner ring image are segmented into four parts equally in the same segmenting manner to obtain four segmented outer ring images and four segmented inner ring images.

Step 106: a four-partitioning intensity difference coefficient of the target thyroid nodule ultrasonic image is obtained according to the intensity average of each segmented outer ring image and the intensity average of each segmented inner ring image.

Step 107: a preprocessed image is input into a trained Thy-Enet deep neural network to obtain the probability that the thyroid nodule boundary of the target thyroid nodule ultrasonic image is clear. The preprocessed image is a target thyroid nodule ultrasonic image in an outwardly expanded target bounding rectangle; the outwardly expanded target bounding rectangle is obtained by outwardly expanding the bounding rectangle of the thyroid nodule boundary labelled in the target thyroid nodule ultrasonic image. The Thy-Enet deep neural network includes an input layer, an EfficientNetV2B0 backbone, a global pooling layer, a dropout layer and a Sigmoid function which are connected in sequence.

Step 108: the aspect ratio coefficient c_{h/w} of the target thyroid nodule ultrasonic image, the inner and outer ring difference coefficient cₒᵤₜ₋ᵢₙ of the target thyroid nodule ultrasonic image, the four-partitioning intensity difference coefficient c_{quar} of the target thyroid nodule ultrasonic image and the probability c_{pred} that the thyroid nodule boundary of the target thyroid nodule ultrasonic image is clear, are input into a trained multi-layer perceptron model to obtain a final probability pₒᵤₜₚᵤₜ that the thyroid nodule boundary of the target thyroid nodule ultrasonic image is clear.

Step 109: a determination result Pred_{final} of the thyroid nodule boundary of the target thyroid nodule ultrasonic image is obtained according to the final probability that the thyroid nodule boundary of the target thyroid nodule ultrasonic image is clear. The determination result is that thyroid nodule boundary is clear or unclear. If the final probability of the thyroid nodule boundary being clear is greater than or equal to 0.5, the nodule boundary is determined to be clear, otherwise, the nodule boundary is determined to be unclear.

In practical application, calculating an aspect ratio coefficient of the target thyroid nodule ultrasonic image according to a width of the bounding rectangle and a height of the bounding rectangle specifically includes:
calculating the ratio of the height of the bounding rectangle to the width of the bounding rectangle to obtain an aspect ratio; and
obtaining the aspect ratio coefficient of the target thyroid nodule ultrasonic image according to the aspect ratio.

In practical application, calculating an inner and outer ring difference coefficient of the target thyroid nodule ultrasonic image according to an intensity average of an outer ring image and an intensity average of an inner ring image specifically includes:
calculating an absolute value of the difference between the intensity average of the outer ring image and the intensity average of the inner ring image to obtain the inner and outer ring difference coefficient of the target thyroid nodule ultrasonic image.

In practical application, segmenting the outer ring image and the inner ring image into four parts equally in the same segmenting manner to obtain four segmented outer ring images and four segmented inner ring images specifically includes the following steps.

The center of the outer ring image is set as an origin; and the outer ring image is segmented into four parts equally in counterclockwise direction according to angle ranges from 0 to 90 degrees, from 90 to 180 degrees, from 180 to 270 degrees and from 270 to 360 degrees, to obtain four segmented outer ring images.

The center of the inner ring image is set as an origin; and the inner ring image is segmented into four parts equally in counterclockwise direction according to angle ranges from 0 to 90 degrees, from 90 to 180 degrees, from 180 to 270 degrees and from 270 to 360 degrees, to obtain four segmented inner ring images.

In practical application, obtaining a four-partitioning intensity difference coefficient of the target thyroid nodule ultrasonic image according to the intensity average of each segmented outer ring image and the intensity average of each segmented inner ring image specifically includes the following steps.

The difference between an intensity average of a first segmented outer ring image and an intensity average of a first segmented inner ring image is calculated to obtain a first difference; where the position of the first segmented outer ring image in the outer ring image is the same as that of the first segmented inner ring image in the inner ring image.

The difference between an intensity average of a second segmented outer ring image and an intensity average of a second segmented inner ring image is calculated to obtain a second difference; where the position of the second segmented outer ring image in the outer ring image is the same as that of the second segmented inner ring image in the inner ring image.

The difference between an intensity average of a third segmented outer ring image and an intensity average of a third segmented inner ring image is calculated to obtain a third difference; where the position of the third segmented outer ring image in the outer ring image is the same as that of the third segmented inner ring image in the inner ring image.

The difference between an intensity average of a fourth segmented outer ring image and an intensity average of a fourth segmented inner ring image is calculated to obtain a fourth difference; where the position of the fourth segmented outer ring image in the outer ring image is the same as that of the fourth segmented inner ring image in the inner ring image.

The minimum value among the first difference, the second difference, the third difference and the fourth difference is determined as the four-partitioning intensity difference coefficient of the target thyroid nodule ultrasonic image.

In practical application, the training process of the Thy-Enet depth neural network includes the following steps.

A sample set is acquired; where the sample set includes a plurality of sample thyroid nodule ultrasonic images and a true probability that the thyroid nodule boundary of each of the sample thyroid nodule ultrasonic images is clear; the thyroid nodule boundary is labelled in the sample thyroid nodule ultrasonic image.

Data enhancement processing is performed on each sample thyroid nodule ultrasonic image in the sample set to obtain a plurality of enhanced sample thyroid nodule ultrasonic images, where one sample thyroid nodule ultrasonic image corresponds to one enhanced sample thyroid nodule ultrasonic image.

The Thy-Enet deep neural network is trained according to the plurality of enhanced sample thyroid nodule ultrasonic images and the true probability that the thyroid nodule boundary of each of the sample thyroid nodule ultrasonic images is clear to obtain the trained Thy-Enet deep neural network.

In practical application, the training process of the multi-layer perceptron model includes the following steps.

A determination result of the thyroid nodule boundary of each of the sample thyroid nodule ultrasonic images is acquired.

The aspect ratio coefficient of each of the sample thyroid nodule ultrasonic images, the inner and outer ring difference coefficient of each of the sample thyroid nodule ultrasonic images, the four-partitioning intensity difference coefficient of each of the sample thyroid nodule ultrasonic images and the probability that the thyroid nodule boundary of each of the sample thyroid nodule ultrasonic images is clear are determined, where the probability that the thyroid nodule boundary of the sample thyroid nodule ultrasonic image is clear is obtained by inputting a preprocessed sample image into the trained Thy-Enet deep neural network; the preprocessed sample image is a sample thyroid nodule ultrasonic image in an outwardly expanded sample bounding rectangle; and the outwardly expanded sample bounding rectangle is obtained by outwardly expanding the bounding rectangle of the thyroid nodule boundary of the sample thyroid nodule ultrasonic image;

The multi-layer perceptron model is trained according to the aspect ratio coefficient of each of the sample thyroid nodule ultrasonic images, the inner and outer ring difference coefficient of each of the sample thyroid nodule ultrasonic images, the four-partitioning intensity difference coefficient of each of the sample thyroid nodule ultrasonic images, the probability that the thyroid nodule boundary of each of the sample thyroid nodule ultrasonic images is clear, and the determination result of the thyroid nodule boundary of each of the sample thyroid nodule ultrasonic images to obtain a trained multi-layer perceptron model.

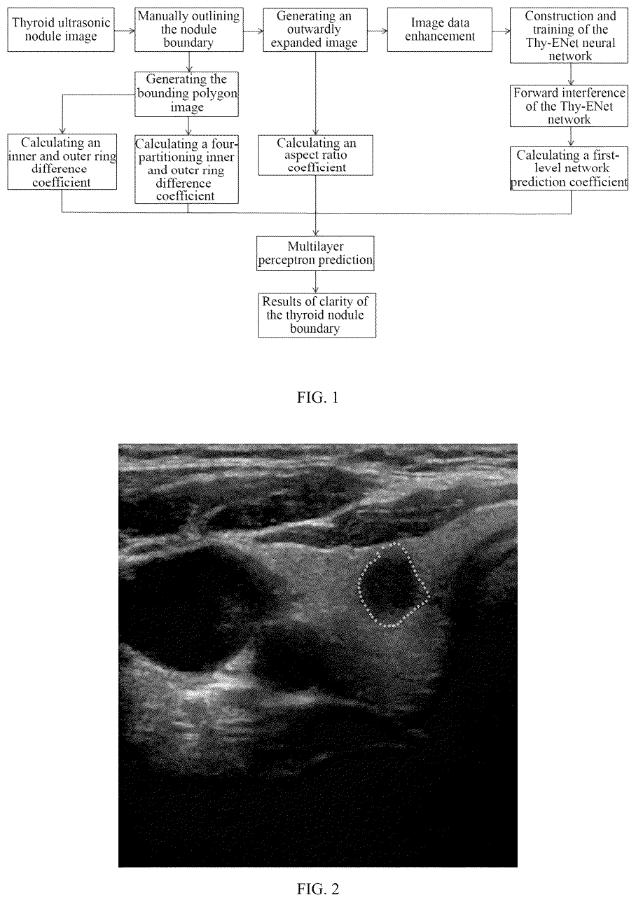

The present disclosure provides a more specific embodiment to introduce the above method in detail, as shown in FIG. 1. The method includes the following steps.

S1: Image preprocessing and enhancement

S1.1: Image preprocessing

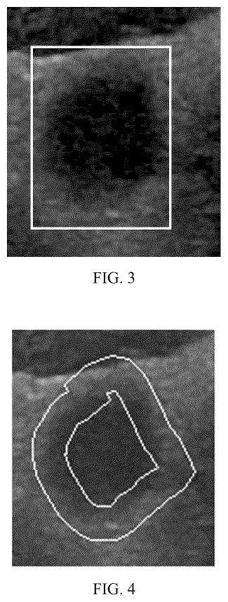

In the embodiment of the present disclosure, 3,127 thyroid nodule ultrasonic images (sample thyroid nodule ultrasonic images) labelled with outline and position of nodule boundary by professional sonographers are used, and the sonographers outline the positions and boundaries of the nodules in the form of dots to form a point set (xᵢ, yᵢ)∈. For any nodule, P contains at least ten outlined points, as shown in FIG. 2. For each nodule image, the doctor labels the nodule boundary as clear or unclear. In the ultrasonic image, a bounding rectangle is obtained for the outlined boundary, as shown in FIG. 3. That is, the positioning bounding rectangle satisfies the equation

$$Rect ( x , y , w , h ) = \{ \begin{matrix}x = min ( {P}_{x} ) \\ y = min ( {P}_{y} ) \\ w = max ( {P}_{x} ) - min ( {P}_{x} ) \\ h = max ( {P}_{y} ) - min ( {P}_{y} )\end{matrix} ,$$

where Rect(x,y,w,h) is the bounding rectangle of the nodule in the image, x and y are the abscissa and the ordinate, respectively, at the upper left corner of the bounding rectangle, w and h are the width and the height, respectively, of the bounding rectangle, Pₓ is a abscissa set of all outlined points, and P_{y} is an ordinate set of all outlined points.

After a basic bounding rectangle is obtained, an outwardly expanded ratio of rectangle is dynamically acquired based on the equation

$$ratio = \{ \begin{matrix}0.4 & if max ( w , h ) < 50 \\ 0.25 & if 50 \leq max ( w , h ) < 100 \\ 0.2 & if 100 \leq max ( w , h ) < 150 \\ 0.12 & if 150 \leq max ( w , h ) < 200 \\ 0.08 & else\end{matrix}$$

according to the size of the bounding rectangle of the nodule, where max (w, h) is the larger value of the width and the height of the rectangle.

Thereafter, the bounding rectangle is outwardly expanded according to the ratio and equation

$${Rect}_{new} ( {x}_{new} , {y}_{new} , {w}_{new} , {h}_{new} ) = \{ \begin{matrix}{x}_{new} = {x}_{old} - ratio * {w}_{old} \\ {y}_{new} = {y}_{old} - ratio * {h}_{old} \\ {w}_{new} = {w}_{old} + 2 * ratio * {w}_{old} \\ {h}_{new} = {h}_{old} + 2 * ratio * {h}_{old}\end{matrix} ,$$

where Rect_{new}(x_{new}, y_{new}, w_{new}, h_{new}) is the outwardly expanded bounding rectangle in the image, x_{new} and y_{new} are the abscissa and the ordinate at the upper left corner of the bounding rectangle, w_{new} and h_{new} are the width and the height of the bounding rectangle, x_{old} and y_{old} are the abscissa and the ordinate at the upper left corner of the bounding rectangle before outward expansion, and Wold and hold are the width and the height of the bounding rectangle before outward expansion. When the outward expansion exceeds the boundary of the image itself, the value of the image boundary is set as the coordinate point of the outwardly expanded boundary. When the outward expansion is completed, all points for outlining the boundary are connected one by one to obtain the bounding polygon of the thyroid nodule boundary. The bounding polygon is outwardly expanded by 30% equidistantly to obtain the outline of the bounding polygon, that is, the outwardly expanded boundary. The bounding polygon is retracted to the center of the polygon by a distance of 30% to obtain the inwardly retracted polygon, that is, the inwardly retracted boundary, as shown in FIG. 4. The bounding rectangular area is intercepted and is scaled to 192×192 pixels using Lanczos interpolation algorithm.

S1.2: Image data enhancement

In order to make the present disclosure have a good effect on ultrasonic images with different brightness, contrast and clarity, as well as nodules with different sizes and types, the following methods are used to enhance the image in the training process. Before inputting image data for each round into the network, one of the following four methods is randomly selected to perform the following online enhancement on the image:

S1.2.1: Random change of brightness:

For an image, the changed pixel intensity value I_{bnew}: I_{bnew}=I_{old}+delta(−thre_{b}≤delta≤thre_{b}) is calculated, and at the same time, it is ensured that I_{bnew}={_{0 if I}_{_{bnew}}_{<0}^{255 if I}^{_{bnew}}^{>255}, where I_{old} is the pixel intensity value of the corresponding pixel before enhancement, delta is the amplitude of brightness change, thre_{b} is 32, and delta is a random integer between −thre_{b} and thre_{b}.

S1.2.2: Random change of contrast

The intensity value I_{cnew}=alpha*I_{old}(1−thre_{c}≤alpha≤1+thre_{c}) of each pixel after the change is calculated, and at the same time, it is ensured that I_{cnew}={_{0 if I}_{_{cnew}}_{<0}^{255 if I}^{_{cnew}}^{>255}, where thre_{c} is 0.3.

alpha is a change ratio value of contrast, and I_{cnew} is a pixel intensity value after the contrast of the image changes.

S1.2.3: Image translation

The image moves randomly in one of the directions of up, down, left and right by threₘ pixels, and 0-intensity pixels are used to supplement the excess part, where threₘ is randomly selected from 0 to 20.

S1.2.4: Image rotation

The image is rotated clockwise or counterclockwise by an angle of threₐ randomly, and 0-intensity pixels are used to supplement the excess part, where threₐ is randomly selected from 0 to 15.

S2: Construction and training of the Thy-Enet deep neural network

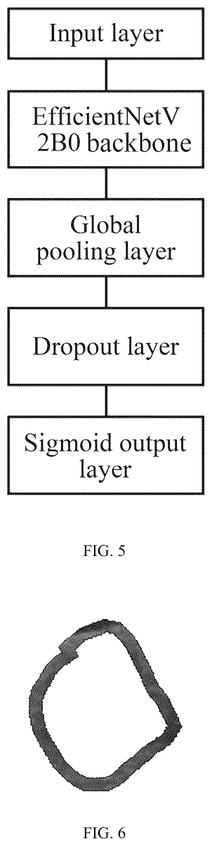

The input size of input layer of the Thy-Enet deep neural network is (w, h, c), where w=192, h=192, c=3. The convolution output in the EfficientNetV2B0 backbone is unfolded by using the global average pooling layer, and then is connected to the dropout layer with a removing probability of 0.5, and is finally connected to the output layer using a Sigmoid activation function, as shown in FIG. 5. An intermediate layer uses leaky ReLU as an activation function. An ultrasonic thyroid nodule image with a size of (192×192×3) is input into the model, and the model outputs the probability that the nodule boundary is clear. 2501 images are extracted as the training set, 313 images are extracted as the validation set, and 313 extracted are extracted as the test set from the 3127 enhanced images. In the training process, an Adam optimizer is used for back propagation of parameters.

S3: Preprocessing the input of the multi-layer perceptron model

S3.1: the aspect ratio of the nodule is calculated, the aspect ratio is calculated according to the width w and the height h of the bounding rectangle obtained in S1.1, T_{h/w}=h/w, and the aspect ratio coefficient

$${c}_{h / w} = \{ \begin{matrix}0 & if {r}_{h / w} \leq 1 \\ 1 & if {r}_{h / w} > 1\end{matrix}$$

is obtained.

S3.2: the intensity difference between the inner ring and the outer ring of the nodule margin is calculated.

According to the method in S1.1, the bounding polygon formed by the nodule boundary outlined by a doctor is outwardly expanded and inwardly retracted in equal proportion, and the intensity average iₒᵤₜ of the outer ring image (as shown in FIG. 6) and the intensity average iᵢₙ of the inner ring image (as shown in FIG. 7) are measured. The inner and outer ring difference coefficient cₒᵤₜ₋ᵢₙ=|iₒᵤₜ−iᵢₙ| is generated.

S3.3: the four-partitioning minimum intensity difference between the inner ring and the outer ring of the nodule margin is calculated.

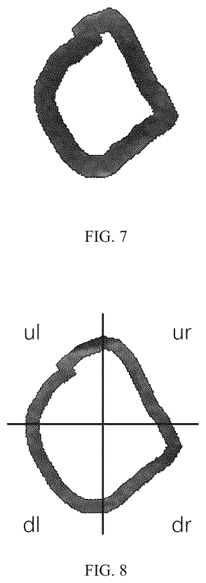

According to the method in S1, after the bounding polygon formed by the nodule boundary outlined by a doctor is outwardly expanded and inwardly retracted in equal proportion, with the center of the image as an origin, the image is segmented into 0-90: ur; 90-180: ul; 180-270: dl; 270-360: dr counterclockwise according to angular coordinates, as shown in FIG. 8. Average intensity differences {dᵤᵣ, dᵤₗ, d_{dl}, d_{dr}} of the outwardly expanded region and inwardly retracted region in the four regions are calculated, and the minimum value of the average intensity differences is determined as the four-partitioning intensity difference coefficient, that is, c_{quar}=min({dᵤᵣ, dᵤₗ, d_{dl}, d_{dr}}).

S3.4: a prediction coefficient of a first-level network is calculated.

The image preprocessed in S1.1 is input into the Thy-Enet deep neural network, and the inference output value of the network is obtained, that is, the probability that the nodule boundary predicted by the Thy-Enet deep neural network is clear, which is denoted as c_{pred}.

S4: the multi-layer perceptron model is constructed and trained.

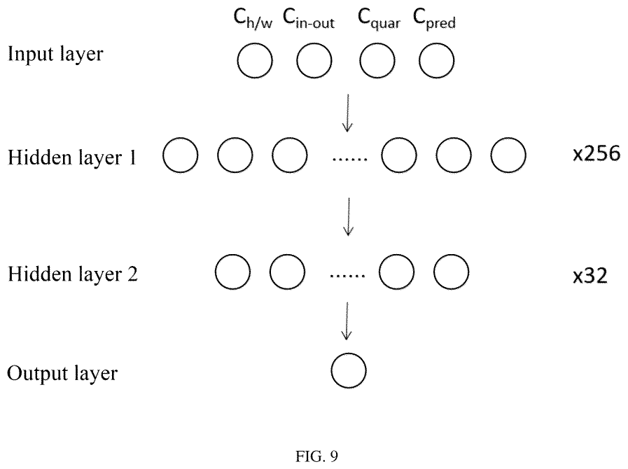

A four-input multi-layer perceptron model is constructed, in which a first layer, including four input units, is an input layer, a second layer, including 256 calculation units, is a hidden layer, the third layer, including 32 calculation units, is a hidden layer, and the fourth layer, including one output unit, is an output layer, as shown in FIG. 9. The hidden layer uses the ReLU activation function, and the output layer uses the sigmoid activation function. The above-mentioned four [c_{h/w} cₒᵤₜ₋ᵢₙ c_{quar} c_{pred}] coefficients are input into the multi-layer perceptron model network. The output result is a floating-point number pₒᵤₜₚᵤₜ between 0 and 1. The final determination result

$${Pred}_{final} = \{ \begin{matrix}{}^{″} {clear}^{″} & if {p}_{output} < {thre}_{p} \\ {}^{″} {unclear}^{″} & if {p}_{output} \geq {thre}_{p}\end{matrix}$$

is obtained according to the floating-point number, where thre_{b} is 0.5. The training set consists of 2501 sets of [c_{h/w} cₒᵤₜ₋ᵢₙ c_{quar} c_{pred}] coefficients corresponding to 2501 images in the training set of the Thy-Enet deep neural network as well as the clear and unclear labels corresponding to the images. The validation set and the test set in the training process also use the coefficients corresponding to the validation set and the test set of the Thy-Enet deep neural network as well as the clear and unclear labels. In the training process, an Adam optimizer is used for back propagation of parameters.

S5: the clarity of the thyroid nodule boundary of the target thyroid nodule ultrasonic image is determined.

For the target thyroid nodule ultrasonic image, first, the doctor labels the thyroid nodule boundary. Thereafter, through the image preprocessing process in S1.1, the input image of the Thy-Enet depth neural network is obtained, and then is input into the trained Thy-Enet depth neural network, to obtain an output c_{pred} predicted by the Thy-Enet depth neural network. Thereafter, according to the method in 3.1, 3.2 and 3.3, c_{h/w}, cₒᵤₜ₋ᵢₙ, c_{quar} and c_{pred} are calculated for this image, and then input into the trained multi-layer perceptron model. Finally, the probability that the thyroid nodule boundary in this image is clear, output by the trained multi-layer perceptron model is obtained. When the probability is greater than or equal to 0.5, the nodule boundary is determined to be clear, otherwise, the nodule boundary is determined to be unclear.

A system for detecting clarity of a thyroid nodule boundary corresponding to the above method according to an embodiment of the present disclosure includes an acquiring module, an aspect ratio coefficient calculating module, a boundary outwardly expanding and inwardly retracting module, an inner and outer ring difference coefficient calculating module, a segmenting module, a four-partitioning intensity difference coefficient calculating module, a clarity probability calculating module, a final clarity probability calculating module and a determination result calculating module.

The acquiring module is configured to acquire a target thyroid nodule ultrasonic image; where a thyroid nodule boundary is labeled in the target thyroid nodule ultrasonic image in a form of dots.

The aspect ratio coefficient calculating module is configured to obtain a bounding rectangle for the thyroid nodule boundary labelled in the target thyroid nodule ultrasonic image, and calculate an aspect ratio coefficient of the target thyroid nodule ultrasonic image according to a width of the bounding rectangle and a height of the bounding rectangle.

The boundary outwardly expanding and inwardly retracting module is configured to outwardly expand and inwardly retract a bounding polygon of the thyroid nodule boundary labelled in the target thyroid nodule ultrasonic image, to obtain an outwardly expanded boundary and an inwardly retracted boundary; where the bounding polygon of the thyroid nodule boundary labelled in the target thyroid nodule ultrasonic image is obtained by connecting all points in the thyroid nodule boundary labelled in the target thyroid nodule ultrasonic image counterclockwise or clockwise in sequence.

The inner and outer ring difference coefficient calculating module is configured to calculate an inner and outer ring difference coefficient of the target thyroid nodule ultrasonic image according to an intensity average of an outer ring image and an intensity average of an inner ring image; where the outer ring image is a first partial target thyroid nodule ultrasonic image between the outwardly expanded boundary and the bounding polygon of the thyroid nodule boundary labelled in the target thyroid nodule ultrasonic image; the inner ring image is a second partial target thyroid nodule ultrasonic image between the inwardly retracted boundary and the bounding polygon of the thyroid nodule boundary labelled in the target thyroid nodule ultrasonic image.

The segmenting module is configured to segment the outer ring image and the inner ring image into four parts equally in a same segmenting manner to obtain four segmented outer ring images and four segmented inner ring images.

The four-partitioning intensity difference coefficient calculating module is configured to obtain a four-partitioning intensity difference coefficient of the target thyroid nodule ultrasonic image according to the intensity average of each segmented outer ring image and the intensity average of each segmented inner ring image.

The clarity probability calculating module is configured to input a preprocessed image into a trained Thy-Enet deep neural network to obtain the probability that the thyroid nodule boundary of the target thyroid nodule ultrasonic image is clear; where the preprocessed image is a target thyroid nodule ultrasonic image in an outwardly expanded target bounding rectangle; the outwardly expanded target bounding rectangle is obtained by outwardly expanding the bounding rectangle of the thyroid nodule boundary labelled in the target thyroid nodule ultrasonic image; the trained Thy-Enet deep neural network includes an input layer, an EfficientNetV2B0 backbone, a global pooling layer, a dropout layer and a Sigmoid function which are connected in sequence.

The final clarity probability calculating module is configured to input the aspect ratio coefficient of the target thyroid nodule ultrasonic image, the inner and outer ring difference coefficient of the target thyroid nodule ultrasonic image, the four-partitioning intensity difference coefficient of the target thyroid nodule ultrasonic image and the probability that the thyroid nodule boundary of the target thyroid nodule ultrasonic image is clear, into a trained multi-layer perceptron model to obtain a final probability that the thyroid nodule boundary of the target thyroid nodule ultrasonic image is clear.

The determination result calculating module is configured to obtain a determination result of the thyroid nodule boundary of the target thyroid nodule ultrasonic image according to the final probability that the thyroid nodule boundary of the target thyroid nodule ultrasonic image is clear; where the determination result is that thyroid nodule boundary is clear or unclear.

The embodiment of the present disclosure further provides an electronic device, including:
a memory and a processor, where the memory is configured to store a computer program, and the processor is configured to run the computer program to cause the electronic device to execute the method for detecting clarity of the thyroid nodule boundary described in the above embodiments.

The embodiment of the present disclosure further provides a computer-readable storage medium, where a computer program is stored therein, and the computer program, when executed by a processor, implements the method for detecting clarity of the thyroid nodule boundary described in the above embodiments.

Compared with the prior art, the present disclosure has the following advantages.

1. Compared with the traditional methods of calculating image indexes such as variance, entropy, and intensity difference of margin, the Thy-Enet depth neural network is used to extract a plurality of depth features related to clarity of the nodule boundary, and image measurement methods are integrated to achieve the goal of multi-dimensional measurement of clarity of the nodule margin. This method has higher accuracy than the existing methods. In addition, this method adapts to ultrasonic images of different quality and sources through image data enhancement, and is less dependent on artificially set thresholds and has a wider applicability.

2. Compared with the method that only uses a deep neural network to predict clarity of the nodule boundary, commonly used indexes such as an aspect ratio and an ultrasonic intensity difference between the inside and the outside of the nodule are designed as the input of multi-layer perceptron model based on the neural network in this method, which increases the interpretability of the method while allowing this method to be more comprehensive and accurate. Compared with other methods based on machine learning, this method uses a large number of images covering a variety of nodules during training, which also ensures that this method can make accurate measurements on different types of nodules.

In this specification, various embodiments are described in a progressive way. The differences between each embodiment and other embodiments are highlighted, and the same and similar parts of various embodiments can be referred to each other. Since the system provided in the embodiment corresponds to the method provided in the embodiment, the device is described simply. Refer to the description of the method for the relevant points.

In the present disclosure, specific examples are applied to illustrate the principle and implementation of the present disclosure, and the explanations of the above embodiments are only used to help understand the method and core ideas of the present disclosure. At the same time, according to the idea of the present disclosure, there will be some changes in the specific implementation and application scope for those skilled in the art. To sum up, the contents of the specification should not be construed as limiting the present disclosure.

What is claimed is:

1. A method for detecting clarity of a thyroid nodule boundary, comprising:
acquiring a target thyroid nodule ultrasonic image, wherein a thyroid nodule boundary is labelled in the target thyroid nodule ultrasonic image in a form of dots;
obtaining a bounding rectangle for the thyroid nodule boundary labelled in the target thyroid nodule ultrasonic image, and calculating an aspect ratio coefficient of the target thyroid nodule ultrasonic image according to a width of the bounding rectangle and a height of the bounding rectangle;
outwardly expanding and inwardly retracting a bounding polygon of the thyroid nodule boundary labelled in the target thyroid nodule ultrasonic image, to obtain an outwardly expanded boundary and an inwardly retracted boundary, wherein the bounding polygon of the thyroid nodule boundary labelled in the target thyroid nodule ultrasonic image is obtained by connecting all points in the thyroid nodule boundary labelled in the target thyroid nodule ultrasonic image counterclockwise or clockwise in sequence;
calculating an inner and outer ring difference coefficient of the target thyroid nodule ultrasonic image according to an intensity average of an outer ring image and an intensity average of an inner ring image, wherein the outer ring image is a first partial target thyroid nodule ultrasonic image between the outwardly expanded boundary and the bounding polygon of the thyroid nodule boundary labelled in the target thyroid nodule ultrasonic image; the inner ring image is a second partial target thyroid nodule ultrasonic image between the inwardly retracted boundary and the bounding polygon of the thyroid nodule boundary labelled in the target thyroid nodule ultrasonic image;
segmenting the outer ring image and the inner ring image into four parts equally in a same segmenting manner to obtain four segmented outer ring images and four segmented inner ring images;
obtaining a four-partitioning intensity difference coefficient of the target thyroid nodule ultrasonic image according to the intensity average of each segmented outer ring image and the intensity average of each segmented inner ring image;
inputting a preprocessed image into a trained Thy-Enet deep neural network to obtain a probability that the thyroid nodule boundary of the target thyroid nodule ultrasonic image is clear, wherein the preprocessed image is a target thyroid nodule ultrasonic image in an outwardly expanded target bounding rectangle, the outwardly expanded target bounding rectangle is obtained by outwardly expanding the bounding rectangle of the thyroid nodule boundary labelled in the target thyroid nodule ultrasonic image, the trained Thy-Enet deep neural network comprises an input layer, an EfficientNetV2B0 backbone, a global pooling layer, a dropout layer and a Sigmoid function which are connected in sequence;
inputting the aspect ratio coefficient of the target thyroid nodule ultrasonic image, the inner and outer ring difference coefficient of the target thyroid nodule ultrasonic image, the four-partitioning intensity difference coefficient of the target thyroid nodule ultrasonic image and the probability that the thyroid nodule boundary of the target thyroid nodule ultrasonic image is clear, into a trained multi-layer perceptron model, to obtain a final probability that the thyroid nodule boundary of the target thyroid nodule ultrasonic image is clear; and
obtaining a determination result of the thyroid nodule boundary of the target thyroid nodule ultrasonic image according to the final probability that the thyroid nodule boundary of the target thyroid nodule ultrasonic image is clear, wherein the determination result is that thyroid nodule boundary is clear or unclear.

2. The method according to claim 1, wherein the calculating an aspect ratio coefficient of the target thyroid nodule ultrasonic image according to a width of the bounding rectangle and a height of the bounding rectangle comprises:
calculating a ratio of the height of the bounding rectangle to the width of the bounding rectangle to obtain an aspect ratio;
obtaining the aspect ratio coefficient of the target thyroid nodule ultrasonic image according to the aspect ratio.

3. The method according to claim 1, wherein the calculating an inner and outer ring difference coefficient of the target thyroid nodule ultrasonic image according to an intensity average of an outer ring image and an intensity average of an inner ring image comprises:
calculating an absolute value of a difference between the intensity average of the outer ring image and the intensity average of the inner ring image to obtain the inner and outer ring difference coefficient of the target thyroid nodule ultrasonic image.

4. The method according to claim 1, wherein the segmenting the outer ring image and the inner ring image into four parts equally in a same segmenting manner to obtain four segmented outer ring images and four segmented inner ring images comprises:
setting a center of the outer ring image as an origin, segmenting the outer ring image into four parts equally in counterclockwise direction according to angle ranges from 0 to 90 degrees, from 90 to 180 degrees, from 180 to 270 degrees and from 270 to 360 degrees, to obtain four segmented outer ring images;
setting a center of the inner ring image as an origin, segmenting the inner ring image into four parts equally in counterclockwise direction according to angle ranges from 0 to 90 degrees, from 90 to 180 degrees, from 180 to 270 degrees and from 270 to 360 degrees, to obtain four segmented inner ring images.

5. The method according to claim 1, wherein the obtaining a four-partitioning intensity difference coefficient of the target thyroid nodule ultrasonic image according to the intensity average of each segmented outer ring image and the intensity average of each segmented inner ring image comprises:
calculating the difference between an intensity average of a first segmented outer ring image and an intensity average of a first segmented inner ring image to obtain a first difference; wherein a position of the first segmented outer ring image in the outer ring image is same as that of the first segmented inner ring image in the inner ring image;
calculating a difference between an intensity average of a second segmented outer ring image and an intensity average of a second segmented inner ring image to obtain a second difference;
wherein a position of the second segmented outer ring image in the outer ring image is same as that of the second segmented inner ring image in the inner ring image;
calculating a difference between an intensity average of a third segmented outer ring image and an intensity average of a third segmented inner ring image to obtain a third difference; wherein a position of the third segmented outer ring image in the outer ring image is same as that of the third segmented inner ring image in the inner ring image;
calculating a difference between an intensity average of a fourth segmented outer ring image and an intensity average of a fourth segmented inner ring image to obtain a fourth difference;
wherein a position of the fourth segmented outer ring image in the outer ring image is same as that of the fourth segmented inner ring image in the inner ring image;
determining a minimum value among the first difference, the second difference, the third difference and the fourth difference as the four-partitioning intensity difference coefficient of the target thyroid nodule ultrasonic image.

6. The method according to claim 1, wherein the trained Thy-Enet depth neural network is determined by a following process:
acquiring a sample set, wherein the sample set comprises a plurality of sample thyroid nodule ultrasonic images and a true probability that a thyroid nodule boundary of each of the sample thyroid nodule ultrasonic images is clear; the thyroid nodule boundary is labelled in each sample thyroid nodule ultrasonic image;
performing data enhancement processing on each sample thyroid nodule ultrasonic image in the sample set to obtain a plurality of enhanced sample thyroid nodule ultrasonic images, wherein one sample thyroid nodule ultrasonic image corresponds to one enhanced sample thyroid nodule ultrasonic image;
training a Thy-Enet deep neural network according to the plurality of enhanced sample thyroid nodule ultrasonic images and the true probability that the thyroid nodule boundary of each of the sample thyroid nodule ultrasonic images is clear to obtain the trained Thy-Enet deep neural network.

7. The method according to claim 6, wherein the trained multi-layer perceptron model is determined by a following process:
acquiring a determination result of the thyroid nodule boundary of each of the sample thyroid nodule ultrasonic images;
determining an aspect ratio coefficient of each of the sample thyroid nodule ultrasonic images, an inner and outer ring difference coefficient of each of the sample thyroid nodule ultrasonic images, a four-partitioning intensity difference coefficient of each of the sample thyroid nodule ultrasonic images and the probability that the thyroid nodule boundary of each of the sample thyroid nodule ultrasonic images is clear; the probability that the thyroid nodule boundary of the sample thyroid nodule ultrasonic image is clear is obtained by inputting a preprocessed sample image into the trained Thy-Enet deep neural network; the preprocessed sample image is a partial sample thyroid nodule ultrasonic image in an outwardly expanded sample bounding rectangle; and the outwardly expanded sample bounding rectangle is obtained by outwardly expanding a bounding rectangle of the thyroid nodule boundary of each sample thyroid nodule ultrasonic image;
training the multi-layer perceptron model according to the aspect ratio coefficient of each of the sample thyroid nodule ultrasonic images, the inner and outer ring difference coefficient of each of the sample thyroid nodule ultrasonic images, the four-partitioning intensity difference coefficient of each of the sample thyroid nodule ultrasonic images, the probability that the thyroid nodule boundary of each of the sample thyroid nodule ultrasonic images is clear, and the determination result of the thyroid nodule boundary of each of the sample thyroid nodule ultrasonic images to obtain the trained multi-layer perceptron model.

8. A system for detecting clarity of a thyroid nodule boundary, comprising:
an acquiring module, which is configured to acquire a target thyroid nodule ultrasonic image; wherein a thyroid nodule boundary is labeled in the target thyroid nodule ultrasonic image in a form of dots;
an aspect ratio coefficient calculating module, which is configured to obtain a bounding rectangle for the thyroid nodule boundary labelled in the target thyroid nodule ultrasonic image, and calculate an aspect ratio coefficient of the target thyroid nodule ultrasonic image according to a width of the bounding rectangle and a height of the bounding rectangle;
a boundary outwardly expanding and inwardly retracting module, which is configured to outwardly expand and inwardly retract a bounding polygon of the thyroid nodule boundary labelled in the target thyroid nodule ultrasonic image, to obtain an outwardly expanded boundary and an inwardly retracted boundary; wherein the bounding polygon of the thyroid nodule boundary labelled in the target thyroid nodule ultrasonic image is obtained by connecting all points in the thyroid nodule boundary labelled in the target thyroid nodule ultrasonic image counterclockwise or clockwise in sequence;
an inner and outer ring difference coefficient calculating module, which is configured to calculate an inner and outer ring difference coefficient of the target thyroid nodule ultrasonic image according to an intensity average of an outer ring image and an intensity average of an inner ring image; wherein the outer ring image is a first partial target thyroid nodule ultrasonic image between the outwardly expanded boundary and the bounding polygon of the thyroid nodule boundary labelled in the target thyroid nodule ultrasonic image; the inner ring image is a second partial target thyroid nodule ultrasonic image between the inwardly retracted boundary and the bounding polygon of the thyroid nodule boundary labelled in the target thyroid nodule ultrasonic image;
a segmenting module, which is configured to segment the outer ring image and the inner ring image into four parts equally in a same segmenting manner to obtain four segmented outer ring images and four segmented inner ring images;
a four-partitioning intensity difference coefficient calculating module, which is configured to obtain a four-partitioning intensity difference coefficient of the target thyroid nodule ultrasonic image according to the intensity average of each segmented outer ring image and the intensity average of each segmented inner ring image;
a clarity probability calculating module, which is configured to input a preprocessed image into a trained Thy-Enet deep neural network to obtain a probability that the thyroid nodule boundary of the target thyroid nodule ultrasonic image is clear; wherein the preprocessed image is a target thyroid nodule ultrasonic image in an outwardly expanded target bounding rectangle; the outwardly expanded target bounding rectangle is obtained by outwardly expanding the bounding rectangle of the thyroid nodule boundary labelled in the target thyroid nodule ultrasonic image; the trained Thy-Enet deep neural network comprises an input layer, an EfficientNetV2B0 backbone, a global pooling layer, a dropout layer and a Sigmoid function which are connected in sequence;
a final clarity probability calculating module, which is configured to input the aspect ratio coefficient of the target thyroid nodule ultrasonic image, the inner and outer ring difference coefficient of the target thyroid nodule ultrasonic image, the four-partitioning intensity difference coefficient of the target thyroid nodule ultrasonic image and the probability that the thyroid nodule boundary of the target thyroid nodule ultrasonic image is clear, into a trained multi-layer perceptron model, to obtain a final probability that the thyroid nodule boundary of the target thyroid nodule ultrasonic image is clear;
a determination result calculating module, which is configured to obtain a determination result of the thyroid nodule boundary of the target thyroid nodule ultrasonic image according to the final probability that the thyroid nodule boundary of the target thyroid nodule ultrasonic image is clear; wherein the determination result is that thyroid nodule boundary is clear or unclear.

9. An electronic device, comprising:
a memory and a processor, wherein the memory is configured to store a computer program, and the processor is configured to run the computer program to cause the electronic device to execute the method for detecting clarity of the thyroid nodule boundary according to claim 1.

10. The electronic device according to claim 9, the processor is configured to run the computer program to cause the electronic device to execute the method of:
calculating a ratio of the height of the bounding rectangle to the width of the bounding rectangle to obtain an aspect ratio;
obtaining the aspect ratio coefficient of the target thyroid nodule ultrasonic image according to the aspect ratio.

11. The electronic device according to claim 9, the processor is configured to run the computer program to cause the electronic device to execute the method of:
calculating an absolute value of a difference between the intensity average of the outer ring image and the intensity average of the inner ring image to obtain the inner and outer ring difference coefficient of the target thyroid nodule ultrasonic image.

12. The electronic device according to claim 9, the processor is configured to run the computer program to cause the electronic device to execute the method of:
setting a center of the outer ring image as an origin, segmenting the outer ring image into four parts equally in counterclockwise direction according to angle ranges from 0 to 90 degrees, from 90 to 180 degrees, from 180 to 270 degrees and from 270 to 360 degrees, to obtain four segmented outer ring images;
setting a center of the inner ring image as an origin, segmenting the inner ring image into four parts equally in counterclockwise direction according to angle ranges from 0 to 90 degrees, from 90 to 180 degrees, from 180 to 270 degrees and from 270 to 360 degrees, to obtain four segmented inner ring images.

13. The electronic device according to claim 9, the processor is configured to run the computer program to cause the electronic device to execute the method of:
calculating the difference between an intensity average of a first segmented outer ring image and an intensity average of a first segmented inner ring image to obtain a first difference; wherein a position of the first segmented outer ring image in the outer ring image is same as that of the first segmented inner ring image in the inner ring image;
calculating a difference between an intensity average of a second segmented outer ring image and an intensity average of a second segmented inner ring image to obtain a second difference; wherein a position of the second segmented outer ring image in the outer ring image is same as that of the second segmented inner ring image in the inner ring image;
calculating a difference between an intensity average of a third segmented outer ring image and an intensity average of a third segmented inner ring image to obtain a third difference; wherein a position of the third segmented outer ring image in the outer ring image is same as that of the third segmented inner ring image in the inner ring image;
calculating a difference between an intensity average of a fourth segmented outer ring image and an intensity average of a fourth segmented inner ring image to obtain a fourth difference; wherein a position of the fourth segmented outer ring image in the outer ring image is same as that of the fourth segmented inner ring image in the inner ring image;
determining a minimum value among the first difference, the second difference, the third difference and the fourth difference as the four-partitioning intensity difference coefficient of the target thyroid nodule ultrasonic image.

14. The electronic device according to claim 9, the processor is configured to run the computer program to cause the electronic device to execute the method of:
acquiring a sample set, wherein the sample set comprises a plurality of sample thyroid nodule ultrasonic images and a true probability that a thyroid nodule boundary of each of the sample thyroid nodule ultrasonic images is clear; the thyroid nodule boundary is labelled in each sample thyroid nodule ultrasonic image;
performing data enhancement processing on each sample thyroid nodule ultrasonic image in the sample set to obtain a plurality of enhanced sample thyroid nodule ultrasonic images, wherein one sample thyroid nodule ultrasonic image corresponds to one enhanced sample thyroid nodule ultrasonic image;
training a Thy-Enet deep neural network according to the plurality of enhanced sample thyroid nodule ultrasonic images and the true probability that the thyroid nodule boundary of each of the sample thyroid nodule ultrasonic images is clear to obtain the trained Thy-Enet deep neural network.

15. The electronic device according to claim 14, the processor is configured to run the computer program to cause the electronic device to execute the method of:
acquiring a determination result of the thyroid nodule boundary of each of the sample thyroid nodule ultrasonic images;
determining an aspect ratio coefficient of each of the sample thyroid nodule ultrasonic images, an inner and outer ring difference coefficient of each of the sample thyroid nodule ultrasonic images, a four-partitioning intensity difference coefficient of each of the sample thyroid nodule ultrasonic images and the probability that the thyroid nodule boundary of each of the sample thyroid nodule ultrasonic images is clear; the probability that the thyroid nodule boundary of the sample thyroid nodule ultrasonic image is clear is obtained by inputting a preprocessed sample image into the trained Thy-Enet deep neural network; the preprocessed sample image is a partial sample thyroid nodule ultrasonic image in an outwardly expanded sample bounding rectangle; and the outwardly expanded sample bounding rectangle is obtained by outwardly expanding a bounding rectangle of the thyroid nodule boundary of each sample thyroid nodule ultrasonic image;
training the multi-layer perceptron model according to the aspect ratio coefficient of each of the sample thyroid nodule ultrasonic images, the inner and outer ring difference coefficient of each of the sample thyroid nodule ultrasonic images, the four-partitioning intensity difference coefficient of each of the sample thyroid nodule ultrasonic images, the probability that the thyroid nodule boundary of each of the sample thyroid nodule ultrasonic images is clear, and the determination result of the thyroid nodule boundary of each of the sample thyroid nodule ultrasonic images to obtain the trained multi-layer perceptron model.

16. A computer-readable storage medium, wherein a computer program is stored therein, and the computer program, when executed by a processor, implements the method for detecting clarity of the thyroid nodule boundary according to claim 1.

17. The computer-readable storage medium according to claim 16, the computer program, when executed by a processor, implements the method of:
calculating a ratio of the height of the bounding rectangle to the width of the bounding rectangle to obtain an aspect ratio;
obtaining the aspect ratio coefficient of the target thyroid nodule ultrasonic image according to the aspect ratio.

18. The computer-readable storage medium according to claim 16, the computer program, when executed by a processor, implements the method of:
calculating an absolute value of a difference between the intensity average of the outer ring image and the intensity average of the inner ring image to obtain the inner and outer ring difference coefficient of the target thyroid nodule ultrasonic image.

19. The computer-readable storage medium according to claim 16, the computer program, when executed by a processor, implements the method of:
setting a center of the outer ring image as an origin, segmenting the outer ring image into four parts equally in counterclockwise direction according to angle ranges from 0 to 90 degrees, from 90 to 180 degrees, from 180 to 270 degrees and from 270 to 360 degrees, to obtain four segmented outer ring images;
setting a center of the inner ring image as an origin, segmenting the inner ring image into four parts equally in counterclockwise direction according to angle ranges from 0 to 90 degrees, from 90 to 180 degrees, from 180 to 270 degrees and from 270 to 360 degrees, to obtain four segmented inner ring images.

20. The computer-readable storage medium according to claim 16, the computer program, when executed by a processor, implements the method of:
calculating the difference between an intensity average of a first segmented outer ring image and an intensity average of a first segmented inner ring image to obtain a first difference; wherein a position of the first segmented outer ring image in the outer ring image is same as that of the first segmented inner ring image in the inner ring image;
calculating a difference between an intensity average of a second segmented outer ring image and an intensity average of a second segmented inner ring image to obtain a second difference; wherein a position of the second segmented outer ring image in the outer ring image is same as that of the second segmented inner ring image in the inner ring image;
calculating a difference between an intensity average of a third segmented outer ring image and an intensity average of a third segmented inner ring image to obtain a third difference; wherein a position of the third segmented outer ring image in the outer ring image is same as that of the third segmented inner ring image in the inner ring image;
calculating a difference between an intensity average of a fourth segmented outer ring image and an intensity average of a fourth segmented inner ring image to obtain a fourth difference; wherein a position of the fourth segmented outer ring image in the outer ring image is same as that of the fourth segmented inner ring image in the inner ring image;
determining a minimum value among the first difference, the second difference, the third difference and the fourth difference as the four-partitioning intensity difference coefficient of the target thyroid nodule ultrasonic image.
